(19) United States
(12) Patent Application Publication
(10) Pub. No.: US 2022/0061944 A1
(43) Pub. Date: Mar. 3, 2022

(54) SELF-REPAIRING MULTILAYER ELASTOMER COMPOSITIONS

(71) Applicant: INEO-TECH SDN BHD, Simpang Amput, Pulau Pinang (MY)

(72) Inventors: Pierre HOERNER, Senlis (FR); Sebastien TREILHES, Gelugor (MY); Philippe SONNTAG, Avon (FR)

(21) Appl. No.: 17/415,390

(22) PCT Filed: Dec. 19, 2019
(86) PCT No.: PCT/MY2019/000049

§ 371 Date: Jun. 17, 2021

(30) Foreign Application Priority Data
Dec. 20, 2018 (MY) PI 2018002814

(51) Int. Cl. A61B 42/10; A41D 19/00; B29C 73/22; B29C 73/16; B29C 35/08; B29C 41/22

(52) U.S. Cl. CPC A61B 42/10; A41D 19/0006; A41D 19/0082; B29C 73/22; B29K 2105/0038; B29C 35/0805; B29C 41/22; A41D 2500/50; B29C 73/163

(57) ABSTRACT

Provided are multilayer elastomer compositions such as multilayer films which possess the ability to self-repair upon puncturing. The multilayer elastomer compositions may be prepared from, for example, styrenic block copolymers and find use in the manufacture of thin walled articles, for example gloves, particularly medical or industrial gloves.

### FIELD

This disclosure generally relates to multilayer elastomer compositions which possess the ability to self-repair upon puncturing. The multilayer elastomer compositions may be prepared from styrenic block copolymers and find use in the manufacture of thin walled articles, for example gloves, particularly medical or industrial gloves.

### BACKGROUND

Rubber gloves are thin elastomeric membranes which act as a barrier to protect the wearer's hands from the external environment.

In the surgical field, this barrier plays a bi-directional role, protecting the patient against the wearer's hand flora, which may contain potentially harmful bacteria, and protecting members of the operative team against biological fluids from the patient, that could carry blood-borne pathogens, such as human immunodeficiency virus (HIV) and hepatitis C virus (HCV).

Conventional surgical gloves, however, present some limitations in providing a consistent physical barrier during use. A surgical glove is a very thin membrane (thickness of only 0.2 mm) that can be easily damaged during use in view of the extreme stress in terms of twisting, pulling and stretching and exposure to body fluids and chemicals. On average, 18% of gloves (range 5-82%) contain minute punctures after use (Guidelines on Hand Hygiene, WHO, 2009, p. 54). Glove breaches undermine the glove's intrinsic ability to act as a barrier to infection. Most glove breaches remain unnoticed and inadvertently expose unwary healthcare workers and patients to a risk of cross-contamination. A recent clinical trial demonstrated that punctured gloves double the risk of surgical site infection (Marti et al. Archives of Surgery, 2009, 144 (6):553-558)

One approach to address this problem is to use two pairs of superposed gloves (double gloving), which can offer increased mechanical protection to the inner glove layer. As a result, the inner layer is expected to be less perforated than the outer layer. The two gloves can also have different colours to visually assist in detecting glove breaches. U.S. Pat. No. 5,224,221 describes a tamper or damage evident surgical glove in the form of a bi-layer glove, comprising an inner layer and an outer layer, in which the outer layer is translucent, in particular yellow, and the inner layer is a contrasting colour, in particular a darker colour such as green or black. If either the inner or outer layer is pierced, liquid can permeate between the two layers. This liquid causes two effects; the colour of the inner layer becomes more apparent through the outer layer, and/or the colour of the liquid becomes apparent through the outer layer.

As an improvement of this indication system, U.S. Pat. No. 9,308,048 discloses gloves, in particular surgical gloves, which have puncture evident characteristics by means of which a wearer can easily identify if the glove has been pierced. These gloves have modified surfaces having an initial contact angle of less than 70 degrees, therefore the aqueous fluid will spread across the surface of the glove sufficiently quickly to effectively indicate to the user that the integrity of the glove has been compromised within an acceptable time period.

However, visual control can be impeded by the presence of staining fluids and biological tissues and residues. Furthermore, wearing two superimposed gloves reduces dexterity, not only because of the double thickness that increases stress on the hand, but also because of the slippage of one layer over the other layer, which both inevitably impair the precision during intricate procedures.

Another approach to the problem is the application of antimicrobial coatings to the elastomeric articles. For example, U.S. Pat. No. 9,149,567 discloses a rubber surgical glove having an inner coating containing antimicrobial agents, such as chlorhexidine gluconate, that can be released onto the skin when the glove is used. With this technology, the antimicrobial chemical is in permanent contact with the wearer's hand and helps to reduce bacterial regrowth during use of the glove. However, as surgical gloves are used each working day for hours, repeated exposure to the antimicrobial chemical under occlusive conditions may promote the selection of bacteria over time, which could result in the development of resistant strains. Also, dermal intolerance (irritation, sensitization) due to occlusion-related effects on the skin barrier (glove-induced perspiration, stratum corneum swelling and post-occlusive barrier impairment) can develop.

To mitigate these risks, alternative technologies have been developed which introduce a disinfecting composition directly inside the glove (not as a coating at the surface), and inside small droplets incorporated in an elastomeric layer which is sandwiched between two boundary layers, as described in U.S. Pat. No. 5,804,628.

U.S. Pat. No. 6,998,158 describes a further improvement of this technology by adjusting the mechanical properties of the external layers (inner and outer) and the size and density of the droplets. The driving force of this concept is to design a mechanism that is set in action in case the glove is perforated by a medical device such as a needle or a scalpel blade. In this case, the disinfecting composition “squirts” directly into the device which helps to reduce the viral load that could be transferred to the glove wearer.

However, this technology was primarily developed to provide added protection in case of percutaneous injuries with infected medical devices, and this squirting mechanism might not be triggered for small glove breaches that occur more frequently and remain unnoticed. Also, in case of such a breach, the disinfecting composition from the droplets can ooze out from the glove and the chemicals can come into contact with the wearer and the patient.

Thus, there remains a need for an elastomeric article, such as a medical glove, which exhibits durable barrier properties during use and good biocompatibility for both the wearer and the patient.

The reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavour to which this specification relates.

### SUMMARY

The present disclosure relates, in general, to multilayer elastomer films which, when punctured, possess the ability to seal the puncture. Such films are particularly useful in the fabrication of articles, such as gloves.

In one aspect the present disclosure provides a multilayer elastomer composition comprising two outer barrier layers (L1 and L3) and one or more inner layers (L2) disposed between the outer barrier layers, wherein at least one inner layer comprises fluid-filled droplets, wherein said fluid-filled droplets comprise a water swellable liquid composition.

Inner Layer (L2)

The inner layer effectively serves as a matrix for the fluid-filled droplets that comprise the water-swellable composition.

The matrix may comprise an elastomer, within which fluid-filled droplets comprising the water-swellable composition are dispersed. The volume fraction of fluid-filled droplets in the matrix may be designated as Fv.

In some embodiments the inner layer comprises an elastomer preferably chosen from the group consisting of natural rubber, polybutadiene, polyisoprene, polychloroprene, polyurethane, acrylic polymers or copolymers, silicone elastomers, SBR (Styrene Butadiene Rubber) copolymers, SBCs (Styrenic Block Copolymers such as SBS, SIS, SEBS, SEEPS), Nitrile Butadiene Rubber copolymers, x-NBR (carboxylated Nitrile Butadiene Rubber) copolymers, butyl rubber, fluoroelastomer, styrene butadiene styrene and blends thereof.

In addition to elastomers, the inner layer may further comprise one or more plasticizer(s) or flexibilizer(s) whose chemical nature and content are compatible with the intrinsic characteristics of the elastomeric materials.

The plasticizer enhances the stretchability and flexibility of the elastomer and preferably comprises liquid, or a mixture of liquid, saturated polyolefins. Preferred plasticizers are mineral oils, but the plasticizer may also be sourced from “green chemistry” for example vegetable oils, such as sunflower, rapeseed, coco oil or others. The plasticizer may also be an oligomer or other elastomer that possesses a sufficient compatibility with the elastomer, such as a low molecular weight polybutadiene, polyisoprene, polyisobutene, amorphous polyolefin copolymers of propylene and ethylene, butyl rubber and other polymers known to have a sufficient compatibility with a rubbery block.

The elastomers of the inner layer may also comprise other conventional additives to provide the required physical aspect and mechanical performance of the finished composition, such as, but not limited to: reinforcing resins and/or fillers, pigments, primary and secondary antioxidants, vulcanization and crosslinking agents, photo-initiators, lubricants, anti-static agents, anti-foam, surfactants, and other processing agents and resins.

In a preferred embodiment, the inner layer L2 comprises a formulated SBC composition, which is processed by dipping using solutions in solvents (solvent casting films).

Water Swellable Liquid Composition

The components of the water swellable liquid composition and the volume fraction Fv of the fluid-filled droplets may be selected as a function of the properties that are desired in the inner layer.

The fluid-filled droplets comprise a liquid diluent and a water swellable polymer. In some embodiments the water swellable polymer comprises superabsorbent polymer. Optionally, the fluid-filled droplets may comprise other additives which improve the swelling speed and/or swelling capacity as well as the gel strength.

The fluid-filled droplets comprising the water swellable polymer may comprise a liquid diluent which is substantially immiscible or immiscible with the elastomer of the inner layer (L2).

By substantially immiscible it may be meant that less than 5% of the liquid diluent is soluble in the elastomer, or less than 4%, or preferentially less than 3%.

The liquid diluent may have a low content of water. In some embodiments the liquid diluent has a water content of less than 12% by weight based on the total weight of the liquid diluent and water. Preferably, less than 10%, or less than 5%, or less than 3%, or less than 1% by weight.

In some embodiments the liquid diluent has a viscosity between about 50 and about 5000 mPa·s. In some embodiments the liquid diluent comprises hydrophilic and hygroscopic properties which prevent premature activation and swelling of the super-absorbent polymer. The liquid diluent may be selected from polyols, organic compounds containing multiple hydroxyl groups, but may be any other liquid which is non-miscible with the elastomer in L2.

The liquid diluent may comprise a single chemical or a blend of chemicals. When the liquid diluent is a polyol it may be selected preferably from polyethylene glycols, glycerine and copolymers of ethylene and propylene glycol of various molecular weights and viscosities. Preferably the diluent is a low to medium viscosity anhydrous polyethylene glycol of a molecular weight between 100 and 1000 Daltons, for example 200 or 400 Daltons.

The water swellable polymer may be any polymeric material that has the ability to absorb and retain large volumes of water or aqueous solutions. As used herein, the term “superabsorbent polymer” is a generic term that covers materials of several classes, such as water absorbing polymers, slush powder, superporous hydrogel (SPHs), which provide superior water absorption in the centre by capillary force due to interconnected structural pores.

These polymers can exist in different forms, for example as raw polymer, but also in other structured forms, such as whiskers, membranes, particles and fibers.

The superabsorbent polymers are preferentially polyelectrolytes, molecules which bear ionic or ionisable moieties and active sites for crosslinking, made from various monomers. They can be classified as “anionic” (or “acidic water absorbing polymer”) or as “cationic” (or “basic water absorbing polymer”).

Anionic superbsorbent polymers may be selected from the group consisting of, but not limited to, polyacrylic acid, poly(lactic acid), saponified vinyl acetate-acrylic ester copolymer, hydrolysed acrylonitrile powder, hydrolysed acrylamide copolymer, ethylene-maleic anhydride copolymer, sulfonated polystyrene, poly(aspartic acid), poly(vinylphosphonic acid), poly(vinylsulfonic acid), poly(vinylphosphoric acid, poly(vinylsulfuric acid) and mixtures thereof.

Cationic superabsorbent polymers may be selected from the group consisting of, but not limited to, polyvinylamine, poly(dialkylaminoalkyl(meth)acrylamide), a polyethylenimine, a polymer prepared from the ester analog of an N-(dialkylamino(meth)acrylamide), a poly 2-dimethylaminoethylmethacrylate, a poly 2-dimethylaminoethylacrylate, a polydiallyldimethylammonium chloride, a poly(vinylguanidine), a poly(allylguanidine), a poly(allylamine), a poly(dimethyldialkylammonium hydroxide), a quaternized poly(meth)acrylamide or ester analog thereof, poly(vinylalcohol-co-vinylamine) and mixtures thereof.

Other agents may also be used. Such agents include a starch (or cellulose) graft acrylonitrile (or acrylic acid, acrylate, methylacrylate, acrylamide, styrene, vinylacetate) copolymer or terpolymer.

A blend of several superabsorbent polymers is also envisaged. In some embodiments the superabsorbent polymer may be in form of a fine powder with an average particle size preferably in the range 0.1 to 20 μm, more preferably in the range of 0.1 to 5 μm

In some embodiments, the fluid-filled droplets may also contain additives to adjust the final characteristics of the composition, such as surfactants and dispersants, dyes, rheological additives, crosslinkers, ionic surfactants and active antimicrobial substances.

The rheological behaviour of the fluid-filled droplet may be adjusted using chemical additives such as rheological additives which may be ionic thickeners or non-ionic polymeric associative thickeners, fibers and preferentially nanocellulose-based or polyacrylic-based reinforcement nanofibers, or swelling clays.

Crosslinkers may be used to provide additional consistency to the liquid diluent and may be selected from the family of macromers of ethylene glycol of different molecular weight and functionality, such as polyethylene glycol dimethacrylate (PEGDA), thiol terminated polyethylene glycol and which may create a 3D network with the diluent. The crosslinking reaction may be triggered, for example, by radiation during sterilization of, for example, a glove, therefore there is no need to add radical initiators.

Ionic surfactants are compounds that may be added to the water swellable composition in order to diminish the sensitivity of the water swelling ability of the superabsorbant to the ionic strength. For example, sodium polyacrylate is made by polymerizing a mixture of sodium acrylate and acrylic acid and the amount of liquid that can be absorbed depends on the ionic strength of the solution. This polymer can absorb 800 times its own weight of distilled water, 300 times its own weight of tap water, but only 60 times its own weight of urine (˜0.9% NaCl). The use of ionic surfactants to counter the effect of ionic strength is described in U.S. Pat. No. 5,274,018. In this patent, the swelling curve of the poly(N-isoproplyacrylamide)(NIPA) gel and sodium dodecyl sulfate (SDS) surfactant was determined in human urine. No significant change in the swelling of the gel was observed from that in pure water, indicating that the swelling power could be preserved under physiological conditions.

Antimicrobial compounds may be added to the water swellable polymer composition and may be preferably chosen from substances capable of causing a virtually instantaneous denaturation of proteins by simple contact, either by chemical reaction or by a physicochemical effect, such as a modification of the surface tension. These may be selected from, quaternary ammonium salts and especially dimethyldidecylammonium chloride, benzalkonium chloride, polydiallyldimethylammonium chloride (Poly DADMAC), biguanides such as water-soluble salts of chlorhexidine, phtalaldehydes, phenolic derivatives such as hexachloroprene or benzyl derivatives, formaldhehyde, non-ionic surfactants comprising at least one polyoxyethylene substance, hexamidine, iodine polyvinylpyrrolidone compounds, and mixture thereof.

According to one particular embodiment of the present disclosure, the intermediate layer L2 may be formed from a superposition of two or more intermediate sublayers each comprising a dispersion of fluid-filled droplets, the nature of the active substances contained in each of the said sublayers being identical or different from one sublayer to another.

In some embodiments the composition of the water swellable composition comprises:

(1) Diluent: 100 parts

(2) Superabsorbent polymers: 1 to 200 parts

(3) Rheological additives: 0 to 25 parts

(4) Crosslinkers: 0 to 20 parts

(5) Antimicrobial compounds: 0 to 50 parts

In some embodiments the volume fraction Fv of the fluid-filled droplets, expressed in the dry film for layer L2 is 1-65%.

As water is typically the “carrier” of potential pathogens (arising either from the inside of a glove or from the outside) the present inventors focused research to develop systems that may use water as the “triggering” agent to induce the repair of physical damage.

A glove made of a multilayer composition that comprises at least one layer containing at least one substance incorporated inside fluid-filled droplets that can quickly swell when in contact with water or biological fluids is provided. In some embodiments the swollen substance will have a volume of at least 20 times its initial state, and this volume expansion fills-up and closes the space generated by, for example, a crack or puncture, thereby acting like an invisible backup system in case of a glove breach.

When the glove is intact, the sweat between the hand and the glove membrane, also known as “glove juice”, remains enclosed inside the glove. This “juice” is likely to contain skin borne bacteria (mainly coagulase negative staphylococcus) originally present on the hand flora of the surgeon. Also, on the outside of the glove, the operating site environment contains biological fluids and blood that can contain various bacteria and blood-borne viruses.

In the event of a glove breach, a small amount of the glove juice could pass through the hole and contaminate the wound. In a reverse process, the biological fluid or blood potentially containing viruses/bacteria from the operating site can also diffuse across a glove through a small breach and contaminate the surgeon's hand.

When the film is damaged, such as by microscopic perforation or abrasion, the substance contained in the fluid-filled droplets is discharged from the droplets located at the vicinity of the site of damage and wicks into the crack. The fluid contained in the droplets is then squashed out in the crack preventing water flowing through the glove. During this process the “water carrier” comes into intimate contact with the composition containing the water-swelling substance, resulting in fast swell, and viscosity and volume increase that will close the small fissure or lesion, preventing any breakthrough of contaminant. The self-repair mechanism preferably occurs within the shortest time after the glove breach.

It has been discovered that the best closure efficiency, as evaluated by the time of action and by the quality of the closing of the micro-fracture, may be obtained when the droplets are filled with a liquid composition containing a fast-swelling polymer powder dispersed in a liquid diluent. The liquid composition is composed of a hygroscopic carrier in which microscopic particles of water-triggered fast-swelling polymer are dispersed. Once the film is breached, the liquid composition resulting from several droplets located at the vicinity of the point of damage has the ability to quickly flow into the crack plane, filling the void with a blend of absorbent particles and low viscosity liquid. The liquid diluent serves therefore as a “carrier” to the polymer, to transport it exactly to the location of the damage. Then, once water is diffusing across the film, the rheology of the liquid composition will significantly increase following the fast swelling of the individual polymer particles in contact with water. Simultaneous to this sharp viscosity increase, the fast swelling of the composition will fill-up the crack, preventing passage of bacteria or viruses from one side of the glove to another.

The viscosity of the liquid diluent should be sufficient to avoid water or blood ingress, as it acts as a first dynamic barrier. Blood has typically a viscosity ranging between 4 and 25 mPa·s at 37° C., therefore the viscosity of the carrier should be higher than this range.

On the other hand, the liquid diluent viscosity should be kept low enough to allow the fluid flow to occur within a short time scale, in the range of one second. The fluid flow can be approximated by the Poiseuille equation (see White, Frank M., (2003) “6”, Fluid Mechanics (5 ed), McGraw-Hill) and should not be too high so as to allow the fluid to be able to flow and fill the crack.

The viscosity of the water swellable composition is strongly affected by the volume fraction of the particles. An example is given in M Krieger and T Dougherty, Trans. Soc. Rheol. 3, 137 (1959).

$$\begin{matrix} Equation \text{1 - The} Krieger \text{-} Dougherry equation \\ \frac{η}{{η}_{0}} = {( 1 - \frac{ϕ}{{ϕ}_{m}} )}^{- [ η ] {ϕ}_{n}} \\ η \text{-} Viscosity of the suspension , \\ {η}_{0} \text{-} viscosity of the medium , \\ ϕ \text{-} Volume fraction of solids in the suspension , \\ {ϕ}_{m} \text{-} maximum packing fraction ( 63 \% for random close packing ) . \\ [ η ] \text{-} Intrinsic viscosity ( 2.5 for spheres ) & \end{matrix}$$

It may be seen from this equation that a small volume fraction variation can induce major viscosity changes, which may hinder fluid flow to the crack plane. Therefore, the amount of water in the liquid diluent should be kept as low as possible. Accordingly, the water content of the liquid diluent is preferably 5% by weight or less.

In order to avoid water vapor sorption by the superabsorbent particles a strongly hygroscopic compound is preferably used as a particle carrier. It was found that anhydrous PEG, which is used as a desiccant for gas fluxes (see for example U.S. Pat. No. 5,471,852), is a suitable material that can avoid swelling of the particles without altering their molecular structure. Furthermore, PEG is a good solvent for most water-soluble compounds and is totally insoluble in the rubber matrix.

In some embodiments desirable properties have been obtained using anhydrous polyethylene glycol of a viscosity of 400 mPa·s as the liquid diluent.

Fast swelling particles are chosen among superabsorbent composites particles. These are lightly crosslinked polymers that swell to a high degree in water or biological fluids. They are widely used in many fields, such as disposable diapers, agriculture, food packaging, artificial snow, and biomedical applications (see, for example, S. Khanlari and M. A. Dube, Polym. Eng. Sci., 55, 1230 (2015); M. T. Nistor, A. P. Chiriac, L. E. Nita, I. Neamtu, and C. Vasile, Polym. Eng. Sci., 53, 2345 (2013); J. Chen and K. Park, J. Control Release, 65, 73 (2000).

The initial swelling rate is important for the present application and compounds will not be selected with regard to their equilibrium swelling rate, as most superabsorbent polymers require a long time, ranging from hours to days to reach equilibrium. The initial swelling is induced by the penetration of water molecules into the polymeric network through diffusion and capillary action (see X. N. Shi, W. B. Wang, and A. Q. Wang, Colloids Surf. B: Biointerfaces, 88, 279 (2011)). In order to have fast swelling particles, a product should be selected that possesses a higher swelling ability, larger surface area, smaller particle size, and lower crosslinking density as reported for example in J. P. Zhang, H. Chen, and A. Q. Wang, Eur. Polym. J., 41, 2434 (2005).

The system containing fast swelling polymer particles dispersed in a liquid diluent has been shown to offer better performance, compared with other alternative systems such as droplets filled with a hydrogel or with a superabsorbent gel for example. In that case, as the starting state is already in a gel form, its viscosity slows down the liquid movement which opposes the water carrier flow therefore allowing some water to ingress into the glove, impairing the quality of closure and the safety of the device. In addition, building a hydrogel or a superabsorbent gel “in-situ” in the droplet would require the use of various chemicals that can affect the biocompatibility of the material. Also, this system would require starting from a liquid composition that contained significant amounts of water which would limit the system efficiency as the water is the trigger for the mechanism of action.

Outer Barrier Layers (L1 and L3)

The outer barrier layers may comprise any elastomeric material.

In some embodiments the outer barrier layers comprise an elastomer preferably chosen from the group consisting of natural rubber, polybutadiene, polyisoprene, polychloroprene, polyurethane, acrylic polymers or copolymers, silicone elastomers, SBR (Styrene Butadiene Rubber) copolymers, SBCs (Styrenic Block Copolymers such as, for example, SBS, SIS, SEBS or SEEPS), Nitrile Butadiene Rubber copolymers, x-NBR (carboxylated Nitrile Butadiene Rubber) copolymers, butyl rubber, fluoroelastomer, styrene butadiene styrene and blends thereof.

It should be understood that the nature of the elastomer(s) comprising each of the said outer layers may be the same or different to each other. In some embodiments, SBCs are preferred elastomers.

In addition to elastomers, the outer barrier layers may further comprise one or more plasticizer(s) or flexibilizer(s) whose chemical nature and content are compatible with the intrinsic characteristics of the elastomeric materials.

The plasticizer enhances the stretchability and flexibility of the elastomer and preferably comprises liquid, or a mixture of liquid, saturated polyolefins. Preferred plasticizers are mineral oils but the plasticizer may also be sourced from “green chemistry”, for example vegetable oils, such as sunflower, rapeseed, coco oil or others. The plasticizer may also be an oligomer or other elastomer that possesses sufficient compatibility with the elastomer, such as a low molecular weight polybutadiene, polyisoprene, polyisobutene, amorphous polyolefin copolymers of propylene and ethylene, butyl rubber and other polymers known to have a sufficient compatibility with a rubbery block.

The elastomers of the outer barrier layers may also comprise other conventional additives to provide the required physical aspect and mechanical performance of the finished composition, such as, but not limited to: reinforcing resins and/or fillers, pigments, primary and secondary antioxidants, vulcanization and crosslinking agents, photo-initiators, lubricants, anti-static agents, anti-foam, surfactants, mold release agents, and other processing agents and resins.

In a preferred embodiment, the outer barrier layers comprise formulated SBC compositions, which are preferentially processed by dipping, using solutions in solvents (solvent cast films).

In another preferred embodiment, the outer layer L1 comprises two superimposed layers, L1a (external) and L1b (in contact with L2). L1b comprises a formulated SBC composition and L1a comprises a chemically crosslinked synthetic rubber, such as polychloroprene, polyisoprene or nitrile butadiene rubber.

In some embodiments the total thickness of the multilayer composition may be between about 10 microns and about 1500 microns, preferably between about 50 microns and about 1000 microns, more preferably between about 100 microns and about 500 microns.

In some embodiments the total thickness of the multilayer composition is less than 1500 microns, or less than 1000 microns, or less than 500 microns.

In some embodiments the thickness e1, e2 and e3 of each of the layers of the material, L1, L2 and L3 respectively may be identical or different, and may independently range between 25 to 500 microns for each of the layers. The elastomers from each layer can be identical or different. The layer L1 may be obtained by superposition of two layers made from different elastomers.

In another aspect the present disclosure provides an article of manufacture, such as a glove, comprising any one of the herein disclosed multi-layer compositions.

In another aspect the present disclosure provides a process for manufacturing a multilayer composition according to any one of the herein disclosed embodiments, said process comprising the following steps:
(a) forming a layer comprising one or more elastomers on a mold;
(b) forming a layer comprising fluid-filled droplets, wherein said fluid-filled droplets comprise a water swellable composition, on top of the layer of one or more elastomers; and
(c) forming a layer comprising one or more elastomers on top of the layer comprising the fluid-filled droplets.

Optionally, one or more further layers may be applied, before or after any one or more of steps (a) and (b) and (c).

The process may be performed by sequentially dipping the mold in a solution of the one or more elastomers.

In some embodiments the layer comprising fluid-filled droplets is formed from an emulsion of the fluid-filled droplets in a liquid solution of elastomer.

In some embodiments the layer comprising fluid-filled droplets is formed from an emulsion of the fluid-filled droplets in a liquid solution of elastomer, wherein the liquid solution of elastomer comprises about 10% to about 30% by weight elastomer.

In some embodiments the emulsion of fluid-filled droplets in a liquid solution of elastomer is prepared by:
(a) forming a solution of elastomer in a suitable solvent;
(b) dispersing a water swellable polymer in a liquid diluent;
(c) adding the dispersion of water swellable polymer to the solution of elastomer to form the emulsion.

In some embodiments the dispersion is facilitated by high shear.

In some embodiments a dispersant is added in step (b) to facilitate dispersion.

In some embodiments step (c) is performed under high shear.

The adhesion between the layers may also be achieved through chemical or physical treatment. Chemical treatment is understood to mean either a grafting or a chemical attack, and physical treatment is understood to mean a bombardment of the surface of the film with ions, electrons (corona or plasma treatment) or photons (ultraviolet treatment).

Gloves also need to be easily donned, therefore the inside of the glove may be modified according to means known in the art such as fibre particles (flock lined gloves), textile fabric (supported gloves), donning without any free powder, such as polymer coating or surface modification (chlorination) or use of powder lubricant.

The article, for example glove, particularly the dried article, may subsequently be exposed to radiation, for example electron beam, gamma, UV or X-Ray radiation.

Further features and advantages of the present disclosure will be understood by reference to the following drawings and detailed description.

### BRIEF DESCRIPTION OF THE DRAWINGS

FIG. 1 is a schematic of a multilayer composition according to one embodiment of the present disclosure.

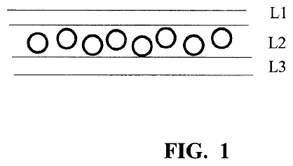

### DETAILED DESCRIPTION OF THE EMBODIMENTS

The following is a detailed description of the disclosure provided to aid those skilled in the art in practicing the present disclosure. Those of ordinary skill in the art may make modifications and variations in the embodiments described herein without departing from the spirit or scope of the present disclosure.

Although any methods and materials similar or equivalent to those described herein can also be used in the practice or testing of the present disclosure, the preferred methods and materials are now described.

It must also be noted that, as used in the specification and the appended claims, the singular forms ‘a’, ‘an’ and ‘the’ include plural referents unless otherwise specified. Thus, for example, reference to a ‘SBC’ may include more than one SBCs, and the like.

Throughout this specification, use of the terms ‘comprises’ or ‘comprising’ or grammatical variations thereon shall be taken to specify the presence of stated features, integers, steps or components but does not preclude the presence or addition of one or more other features, integers, steps, components or groups thereof not specifically mentioned.

Unless specifically stated or obvious from context, as used herein, the term ‘about’ is understood as within a range of normal tolerance in the art, for example within two standard deviations of the mean. ‘About’ can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein in the specification and the claim can be modified by the term ‘about’.

Any methods or processes provided herein can be combined with one or more of any of the other methods or processes provided herein.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims.

### EXAMPLE

The following example demonstrates the improved performance (self-repairing when in contact with water) of a multilayer composition according to the present disclosure.

In this example, the glove is made of three layers, L1, L2 and L3.

Layers 1 and 3

The elastomer composition of Layers 1 and 3 is made of Styrenic Block Copolymer (SBC) and more specifically SEBS with a radial structure containing 31% of polystyrene and a viscosity of 75cp in toluene at 5% concentration.

The elastomer is blended with a resin made of styrene and substituted styrene (Mn=800 g/mol, polydispersity index=2.8), and a white mineral oil with a viscosity of 68 mPas at 40° C. as a plasticizer.

These components are dissolved in a mixture of methylcyclohexane and toluene (8:2) to form a solution having 18% solid content by weight.

The amount of resin is 20 phr, and the amount of plasticizer is 60 phr.

The solution designated as “L1/3-MIX” is stored at ambient temperature in an appropriate vessel covered to prevent solvent evaporation.

Layer 2

The elastomeric ingredients used for Layer 2 are similar than those used for the Layers 1 and 3 described above. For Layer 2, the amount of resin is 10 phr and the amount of plasticizer is 45 phr.

These components are dissolved in a mixture of methylcyclohexane and toluene (8:2) to form a solution having 18% solid content by weight. This solution is designated “continuous phase”.

The water swellable composition is composed of polyethylene glycol (molecular weight of 400 g/mol) mixed with a cationic polymer particle (superabsorbent polymer) made of poly 2 dimethylaminoethylmethacrylate.

The quantity of superabsorbent polymer is 10% compared to the polyethylene glycol 400.

The dispersion of superabsorbent polymer is performed under high shear to allow a proper particle dispersion in the presence of benzalkonium chloride that serves as a dispersant (5% of the total composition). This liquid phase is designated “dispersed phase”.

Under shear, the “dispersed phase” is gently added into the “continuous phase” in order to obtain a final emulsion, designated “L2-MIX” which is stored under light shearing to prevent destabilization and sedimentation. The volume fraction Fv of the droplets, expressed in the dry film (that is, after evaporation of the solvent) is 40%.

Multilayer films were obtained following solvent evaporation after dipping a porcelain glove mould into the “L1/3-Mix” first, using a dipping robot with controlled dipping speeds.

The film is allowed to dry at 40° C. for 15 minutes before performing another dipping in the same mixture, following by another drying of 15 minutes at 40° C. The Layer 1 is the result of these two thin layers.

Layer 2 is then applied on top of Layer 1 by dipping in the “L2-MIX”. A continuous film incorporating the water composition inside the droplets is formed upon evaporation of the solvent.

The film is then allowed to dry at 40° C. for 30 minutes before depositing Layer 3.

Layer 3 is deposited by dipping in the “L1/3-MIX”, then the film is allowed to dry first at 40° C. during 15 minutes before a final drying at 75° C. for 1 hour. After stripping, the film is placed in an oven at 60° C. for 6 hours to remove trace amounts of residual solvent.

The film is packed in a pouch consisting of a thin layer of aluminium, a metal that has very high oxygen and moisture barrier properties (therefore rendering extremely difficult for water to pass through the film) then exposed to electron beam radiation at a dose of 25±2 kGy.

A small glove breach is simulated in a glove finger using a 0.3 mm acupuncture needle, then the finger is gently filled with colored water. Application of minor pressure on the finger facilitates the water passing across the film through the hole. Almost immediately after contact with water, the hole is filled with the gel formed by the superabsorbent polymer, stopping the passage of water.

The contents of all references, and published patents and patent applications cited throughout the application are hereby incorporated by reference. Those skilled in the art will recognize that the disclosure may be practiced with variations on the disclosed structures, materials, compositions and methods, and such variations are regarded as within the ambit of the disclosure.

Those skilled in the art will recognize, or be able to ascertain using no more than routine experimentation, many equivalents to the specific embodiments and methods described herein. Such equivalents are intended to be encompassed by the scope of the following claims.

It is understood that the detailed examples and embodiments described herein are given by way of example for illustrative purposes only, and are in no way considered to be limiting to the disclosure. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described.

Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

1. A multilayer elastomer composition comprising two outer barrier layers (L1 and L3) and one or more inner layers (L2) disposed between the outer barrier layers, wherein at least one inner layer comprises fluid-filled droplets, wherein said fluid-filled droplets comprise a water swellable liquid composition.

2. A multilayer elastomer composition according to claim 1, comprising one or more further layers.

3. A multilayer elastomer composition according to claim 1, wherein the fluid-filled droplets comprise a liquid diluent which is substantially immiscible with the elastomer of the inner layer.

4. A multilayer elastomer composition according to claim 3, wherein the liquid diluent comprises less than 12% by weight water.

5. A multilayer elastomer composition according to claim 1, wherein the water swellable liquid composition comprises superabsorbent polymer.

6. A multilayer elastomer composition according to claim 1, wherein the each of layers L1, L2 and L3, independently comprise elastomer selected from the group consisting of natural rubber, polybutadiene, polyisoprene, polychloroprene, polyurethane, acrylic polymers or copolymers, silicone elastomers, SBR (Styrene Butadiene Rubber) copolymers, SBCs (Styrenic Block Copolymers such as, for example, SBS, SIS, SEBS or SEEPS), Nitrile Butadiene Rubber copolymers, x-NBR (carboxylated Nitrile Butadiene Rubber) copolymers, butyl rubber, fluoroelastomer, styrene butadiene styrene and blends thereof.

7. A multilayer elastomer composition according to claim 6, wherein the SBC independently comprises one or a mixture of SBCs of number average molecular weight (Mn) above 100,000 g/mol.

8. A multilayer elastomer composition according to claim 1, wherein the layers independently comprise one or more plasticizers.

9. A multilayer elastomer composition according to claim 8, wherein the plasticizer comprises a liquid, or a mixture of liquid, saturated polyolefins compatible with the midblock (elastomeric block) of an SBC.

10. A multilayer elastomer composition according to claim 8, wherein the plasticizer is selected from one or more vegetable oils, such as sunflower, rapeseed, or coconut oil or an oligomer or other elastomer that possesses sufficient compatibility with the rubbery mid-blocks, such as a low molecular weight polybutadiene, polyisoprene, polyisobutene, amorphous polyolefin copolymers of propylene and ethylene, and butyl rubber.

11. (canceled)

12. A multilayer elastomer composition according to claim 1, wherein the outer layer L1 comprises two superimposed layers, L1a (external) and L1b (in contact with L2).

13. A multilayer elastomer composition according to claim 12, wherein L1b comprises a formulated SBC composition and L1a comprises a chemically crosslinked synthetic rubber, such as polychloroprene, polyisoprene or nitrile butadiene rubber.

14. A multilayer elastomer composition according to claim 3, wherein the liquid diluent has a viscosity between about 50 and about 5000 mPa·s.

15. A multilayer elastomer composition according to claim 3, wherein the liquid diluent is one or more polyols.

16. (canceled)

17. A multilayer elastomer composition according to claim 5, wherein the superabsorbent polymer is an anionic superbsorbent polymer selected from the group consisting of polyacrylic acid, poly(lactic acid), saponified vinyl acetate-acrylic ester copolymer, hydrolysed acrylonitrile powder, hydrolysed acrylamide copolymer, ethylene-maleic anhydride copolymer, sulfonated polystyrene, poly(aspartic acid), poly(vinylphosphonic acid), poly(vinylsulfonic acid), poly(vinylphosphoric acid, poly(vinylsulfuric acid) and mixtures thereof.

18. A multilayer elastomer composition according to claim 5, wherein the superabsorbent polymer is a cationic superabsorbent polymers selected from the group consisting of polyvinylamine, poly(dialkylaminoalkyl(meth)acrylamide), a polyethylenimine, a polymer prepared from the ester analog of an N-(dialkylamino(meth)acrylamide), a poly 2-dimethylaminoethylmethacrylate, a poly 2-dimethylaminoethylacrylate, a polydiallyldimethylammonium chloride, a poly(vinylguanidine), a poly(allylguanidine), a poly(allylamine), a poly(dimethyldialkylammonium hydroxide), a quaternized poly(meth)acrylamide or ester analog thereof, poly(vinylalcohol-co-vinylamine) and mixtures thereof.

19. A multilayer elastomer composition according to claim 5, wherein the superabsorbent polymer has a particle size in the range 0.1 to 20 micron, preferably 0.1 to 5 micron.

20. A multilayer elastomer composition according to claim 1, wherein the water swellable liquid composition further comprises an anionic surfactant.

21. A multilayer elastomer composition according to claim 1, wherein the water swellable liquid composition further comprises one or more crosslinkers.

22. (canceled)

23. A multilayer elastomer composition according to claim 1, wherein the volume fraction of the fluid-filled droplets expressed in the dry film for layer L2 is from 1 to 65%.

24. A multilayered elastomer composition according to claim 1, wherein the total thickness of the multilayer composition is between about 10 microns and about 1500 microns, preferably between about 50 microns and about 1000 microns, more preferably between about 100 microns and about 500 microns.

25. (canceled)

26. An article of manufacture comprising a multilayer elastomer composition according to claim 1 wherein the article is a glove.

27. (canceled)

28. (canceled)

29. (canceled)

30. (canceled)

31. (canceled)

32. (canceled)

33. (canceled)
